SEQUENCE LISTING (6 sequences)

SEQ ID NO: 1 - DNA, 154 bp, ARTIFICIAL SEQUENCE
  OLIGONUCLEOTIDE
ccggctcttccagagcaaggcaaccacaggagaccctgtcacgtcctgcacgacctcttccagagcaaggcaaccacaggagaccctgtcacgtcctgcacgacctcttccagagcaaggcaaccacaggagaccctgtcacgtcctgcacgac

SEQ ID NO: 2 - DNA, 154 bp, ARTIFICIAL SEQUENCE
  OLIGONUCLEOTIDE
gagaaggtctcgttccgttggtgtcctctgggacagtgcaggacgtgctggagaaggtctcgttccgttggtgtcctctgggacagtgcaggacgtgctggagaaggtctcgttccgttggtgtcctctgggacagtgcaggacgtgctgggcc

SEQ ID NO: 3 - DNA, 136 bp, ARTIFICIAL SEQUENCE
  OLIGONUCLEOTIDE
ccggggtgtgcatttagctaaattccccactgtcacgtcctgcacgacggtgtgcatttagctaaattccccactgtcacgtcctgcacgacggtgtgcatttagctaaattccccactgtcacgtcctgcacgac

SEQ ID NO: 4 - DNA, 136 bp, ARTIFICIAL SEQUENCE
  OLIGONUCLEOTIDE
ccacacgtaaatcgatttaaggggtgacagtgcaggacgtgctgccacacgtaaatcgatttaaggggtgacagtgcaggacgtgctgccacacgtaaatcgatttaaggggtgacagtgcaggacgtgctgggcc

SEQ ID NO: 5 - DNA, 192 bp, ARTIFICIAL SEQUENCE
  OLIGONUCLEOTIDE
ccggctcttccagagcaaggcaaccacaggagaccctgtcacgtcctgcacgacggtgtgcatttagctaaattccccactgtcacgtcctgcacgacctcttccagagcaaggcaaccacaggagaccctgtcacgtcctgcacgacggtgtgcatttagctaaattccccactgtcacgtcctgcacgac

SEQ ID NO: 6 - DNA, 192 bp, ARTIFICIAL SEQUENCE
  OLIGONUCLEOTIDE
gagaaggtctcgttccgttggtgtcctctgggacagtgcaggacgtgctgccacacgtaaatcgatttaaggggtgacagtgcaggacgtgctggagaaggtctcgttccgttggtgtcctctgggacagtgcaggacgtgctgccacacgtaaatcgatttaaggggtgacagtgcaggacgtgctgggcc

(19) United States
(12) Patent Application Publication
(10) Pub. No.: US 2013/0236433 A1
(43) Pub. Date: Sep. 12, 2013

(54) METHODS, COMPOSITIONS, CELLS, AND KITS FOR TREATING ISCHEMIC INJURY

(71) Applicant: Keith A. Webster, Key Biscayne, FL (US)

(72) Inventor: Keith A. Webster, Key Biscayne, FL (US)

(21) Appl. No.: 13/884,057

(22) PCT Filed: Nov. 10, 2011
(86) PCT No.: PCT/US11/60103

§ 371 Date: May 29, 2013

Related U.S. Application Data
(60) Provisional application No. 61/412,528, filed on Nov. 11, 2010.

(51) Int. Cl. A61K 35/28; A61K 31/7088; A61K 35/12

(52) U.S. Cl. CPC A61K 35/28; A61K 35/12; A61K 31/7088
USPC 424/93.7

(57) ABSTRACT

The methods, compositions, cells and kits described herein are based on the discovery that stem cells, when injected into ischemic tissue of mammals, can be protected by preconditioning of the ischemic tissue with hypoxia-regulated human VEGF and human IGF-1. Methods, compositions, cells and kits for treating tissue injured by ischemia or at risk of ischemic injury in a subject are thus described herein.

### CROSS-REFERENCE TO RELATED APPLICATIONS

This application claims the benefit of Provisional Application Ser. No. 61/412,528 filed Nov. 11, 2010, which is herein incorporated by reference in its entirety.

### FIELD OF THE INVENTION

The invention relates generally to the fields of medicine, cellular therapy and gene therapy. More particularly, the invention relates to composition, cells, methods and kits for preventing or treating ischemic injury by providing at least one cell survival factor and stem cells to a subject suffering from or at risk of ischemic injury (e.g., patients with diseases such as peripheral artery disease (PAD) and coronary artery disease (CAD)).

### BACKGROUND

Several different populations of stem cells have been shown to increase perfusion and improve function of ischemic skeletal and cardiac muscles in vivo in animal and human subjects. CD34+ endothelial progenitor cells have the capacity to induce neo-angiogenesis and promote reperfusion and function of ischemic myocardium and lower limbs (Dzau V J, et al. Hypertension 2005; 46:7-1; Tateishi-Yuyama E, et. Al., Lancet. 2002, 360:427-35; Van Huyen J P, et al. Mod Pathol. 2008, 21:837-46). Bone-marrow or adipose-derived mesenchymal stem cells (MSCs) can differentiate into multiple cell types including cardiac myocytes and endothelial cells, and secrete reparative cytokines and growth factors. These cells provide an alternative population to endothelial progenitor cells (EPCs) for cell therapy of ischemic organs including myocardial and limb muscle. A major limitation to the efficacy of MSC therapy is the poor viability of the transplanted cells. It has been reported that MSC therapy for the treatment of ischemic organ failure including kidney, heart, and limbs is severely limited because of cell survival within the toxic environment of the ischemic tissue (Dzau, V J, Gnecchi, M., Pachori, A S. J. Am. Coll. Cardiol., 2005; 46:1351-1353; Tang et al, J. Am. Coll. Cardiol., 2005; 46:1339-1350). For example, intravenous delivery of MSCs was reported to produce maximal cell transplantation between days 0-2 after delivery but fell to less than 1% in lung; less than 5% in kidney and about 20% in liver at Day 7, (Volker et al, Exp. Nephrol., Vol. 114, No. 3, 2010). The survival of human MSCs delivered by intra-cardiac injection of infarcted myocardium in SCID mice was reported to be 0.44% at 4 days post-injection (n=12) (Toma et al, Circulation, 2002; 105:93-98). Hoffmann et al. reported close to zero survival of MSCs at 6-days post-injection of ischemic limbs (Thorac Cardiovasc Surg 2010; 58(3): 136-142). Whereas MSC engineering has been shown to improve survival and performance in ischemic hearts (Mangi et al, Nat. Med, 9:1195-9, 2003; Tang et al, J. Am. Coll. Cardiol., 2005; 46:1339-1350), the engineered cells expressing permanent survival factors may pose additional risk to therapy including increased risk of oncogenic transformation.

Accordingly, improved methods of treating ischemic injury with therapeutic stem cells are needed.

### SUMMARY

Described herein are compositions, cells, kits and methods that include use of hypoxia-regulated, and/or inflammation-responsive conditionally-silenced nucleic acids to promote stem cell survival and arteriogenesis in the setting of ischemic disease in a subject (e.g., human patient) that can include peripheral and coronary artery diseases as well as other diseases involving ischemia. To address the problems associated with delivery of stem cells to ischemic tissue, it was hypothesized that tissue engineering with hypoxia-regulated growth and survival factors before cell therapy may reduce toxicity, promote cell survival, and improve therapy. To this end, a rabbit ischemic hind limb model was used to test the effects of tissue engineering with hypoxia-regulated Adeno-associated virus 9 (AAV9) expressing VEGF alone or VEGF±IGF-1 under the direction of a tightly regulated, conditionally silenced promoter (containing FROG and TOAD silencer elements described in Malone et al, Proc Natl Acad Sci. 94, 12314-9, 1997) followed by injection of MSCs. The results indicate significantly improved cell survival and tissue reperfusion using this combination of gene therapy and stem cell therapy.

A nucleic acid (e.g., a DNA vector) that expresses a gene product (i.e., a gene product that protects stem cells in an ischemic environment) under the direction of a hypoxia-regulated, and/or inflammation-responsive conditionally-silenced (CS) promoter is delivered to a tissue that is or may become ischemic. Stem cells that may have therapeutic value delivered to the same tissue are protected from ischemia by the hypoxia-activated (and/or inflammation-activated) gene product of the DNA vector. The combined therapy induces directional growth of blood vessels and arteriogenesis. Ischemic tissue constitutes a toxic environment wherein host cells can become necrotic or apoptotic. As a consequence, when potentially therapeutic cells are injected into sites of ischemia they have shown poor survival; this situation has heretofore limited stem cell therapy for ischemic disease. Described herein is a strategy to address this situation and to protect stem cells when injected into ischemic tissue by preconditioning the tissues with a hypoxia-regulated gene product that is protective (e.g., human vascular endothelial growth factor (h-VEGF) and insulin-like growth factor-1 (h-IGF-1)) contained in a delivery vehicle (e.g., a viral vector such as a semi-permanent AAV delivery vehicle). VEGF and IGF-1 are well-characterized cell survival factors and their expression must be tightly regulated to prevent possible oncogenesis or stimulation of cell survival and proliferation where it is not needed. To test for interactions between injected AAV-CS-VEGF-IGF-1 (see FIG. 4) and stem cell therapy, rabbit (and mouse) hind limbs were injected with AAV-CS-VEGF-IGF-1 (or control PBS). Two weeks later, the limbs were made ischemic by ligation and excision of the femoral artery, and after a further 24 h, syngenic bone marrow mesenchymal stem cells labeled with fluorescent Dil were injected. After 5 more days, rabbits were sacrificed and muscle was collected in the region of ischemia+transgene (experimental) or stem cells only (controls). Stem cell survival was quantified in muscle sections by confocal microscopy. Significantly greater stem cell survival (p<0.01; n=6) was found in the limbs that were pretreated with AAV-CS-VEGF-IGF-1 (see FIG. 1). A mouse ischemic hind limb model was used to monitor safety, regulation of gene expression and restriction of VEGF expression to ischemic muscle. Conditions were the same as in the rabbit model wherein gene therapy was implemented followed by stem cell injections. It was found that hVEGF expression after induction of ischemia peaked at 100-fold more than that in non-ischemic tissue during the first 7 days of ischemia. Subsequently, expression of hVEGF declined to the control levels found in normoxic (nonischemic) tissue. The decline in hVEGF expression correlated with reperfusion of the ischemic tissue assessed by laser Doppler flow measurements in the thigh and ankle regions. To determine long-term safety mice were injected with 1× and 10× doses of AAV-CS-VEGF and tissues were examined after >1 year (lifespan equivalent of 30 human years) for pathology, tumors and vessel growth. Pathological examination indicated no evidence of injury or tumorigenesis in any tissues with either dose. Vessels stained with fluorescent Dil revealed regeneration of the entire femoral artery in limbs that were injected with AAV-CS-hVEGF, but not in limbs that were injected with PBS or unregulated AAV-hVEGF. It is concluded that this protocol that includes gene therapy followed by stem cell therapy is safe and promotes stem cell survival and arteriogenesis. In other experiments described in the Examples below, it was found that the degree of regulation of the AAV-VEGF-IGF-1 by ischemia contributed to the level of tissue and cell protection. Tight regulation of the AAV in multiple cell types (somatic, stem, neuronal) was conferred by 3 silencer elements including Neural Responsive Silencer Element (NRSE), FROG, TOAD in combination with Hypoxia Responsive Element (HREs, also referred to as Hypoxia Responsive Enhancers) (see FIG. 4). AAV expressing hVEGF containing these 3 silencers provided significantly superior cell survival and tissue salvage than the same AAV that contained only one (NRSE) silencer type.

Gene therapy using hypoxia (and/or inflammation)-regulated, conditional silenced AAV vectors with one, two or more (e.g., 3, 4, 5) heterologous silencer elements prior to stem cell therapy is a novel approach to optimize cellular therapy. Conditional silencing with multiple silencer elements provides optimal tissue engineering by gene silencing in all cell types (somatic, stem, neuronal), containment of the foreign gene product within the ischemic tissue and optimization of angiogenesis and vasculogenesis in that region. AAV without sufficient regulation does not efficiently achieve these goals.

Accordingly, a method of treating tissue injured by ischemia or at risk of ischemic injury in a subject is described herein. The method includes the steps of: administering to the subject a therapeutically effective amount of a composition including at least one nucleic acid encoding at least one cell survival factor (e.g., VEGF, FGF, IGF-1, PDGF, and HIF-1) for protecting one or more cell types of: somatic cells, stem cells, and progenitor cells, from ischemia in the subject, the at least one nucleic acid operably linked to a hypoxia-regulated promoter; and administering to the subject a therapeutically effective amount of a plurality of at least one of: somatic cells, stem cells, and progenitor cells. Administering the at least one nucleic acid followed by administration of the plurality of at least one of: somatic cells, stem cells, and progenitor cells induces directional growth of blood vessels and arteriogenesis at one or more sites of ischemia, ischemic injury, and potential ischemic injury in the subject. The at least one cell survival factor can be, e.g., human VEGF (hVEGF). The at least one nucleic acid can further encode a second cell survival factor, e.g., human IGF-1 (hIGF-1). The at least one nucleic acid can be within a recombinant Adeno-Associated Virus (rAAV) vector. In the method, the subject typically has ischemia or ischemia-related disease (e.g., PAD, CAD, ischemic heart disease, and heart failure). The tissue can be, for example, cardiac or skeletal tissue. In one embodiment, the tissue is infracted myocardium and the plurality of at least one of: somatic cells, stem cells, and progenitor cells is delivered by intra-cardiac injection. The plurality of at least one of: somatic cells, stem cells, and progenitor cells can include MSCs. The hypoxia-regulated promoter can be a conditionally silenced promoter (e.g., a hypoxia-regulated promoter conditionally silenced by a Neuronal Response Silencer Element (NRSE) and a Hypoxia Responsive Element (HRE); by FROG and an HRE; by TOAD and an HRE; by FROG, TOAD, and an HRE; by one or more combinations of: NRSE and HRE; FROG and HRE; TOAD and HRE; by FROG, TOAD and HRE, etc.). The hypoxia-regulated conditionally silenced promoter can include at least one of: a metal response element (MRE) and an HRE, and optionally an inflammatory responsive element (IRE). In some embodiments, the hypoxia-regulated conditionally silenced promoter includes an HRE, an MRE, and an IRE, and is responsive to both hypoxia and inflammation.

In the method, the at least one of stem cells and progenitor cells are MSCs obtained from at least one of: bone marrow, adipose, endothelial progenitor cells, CD34+ cells, hematopoietic cells, cardiac myoblasts, skeletal myoblasts, cardiac stem cells, skeletal stem cells, satellite cells, fibroblasts, myofibroblasts, smooth muscle cells, embryonic stem cells, and adult stem cells. The tissue injured by ischemia or at risk of ischemic injury can be, for example, skeletal muscle, cardiac muscle, kidney, liver, dermal tissue, scalp, and eye.

Also described herein is a method of treating tissue injured by ischemia or at risk of ischemic injury in a subject. The method includes the steps of: administering to the subject a therapeutically effective amount of a composition comprising at least one nucleic acid encoding at least one cell survival factor for protecting one or more cell types selected from the group consisting of: somatic cells, stem cells, and progenitor cells, from ischemia in the subject, the at least one nucleic acid operably linked to an inflammation-responsive promoter; and administering to the subject a therapeutically effective amount of a plurality of at least one of: somatic cells, stem cells, and progenitor cells. In the method, the inflammation-responsive promoter can include at least one IRE. The inflammation-responsive promoter can be also responsive to hypoxia (ischemia). Administering the at least one nucleic acid followed by administration of the plurality of at least one of: somatic cells, stem cells, and progenitor cells induces directional growth of blood vessels and arteriogenesis at one or more sites of ischemia, ischemic injury, and potential ischemic injury in the subject.

Further described herein is a kit for treating tissue injured by ischemia or at risk of ischemic injury in a mammalian subject. The kit includes: a therapeutically effective amount of a composition including at least one nucleic acid encoding at least one cell survival factor for protecting at least one of somatic cells, stem cells and progenitor cells from ischemia in the subject, the at least one nucleic acid operably linked to a hypoxia-regulated promoter; a therapeutically effective amount of the at least one of somatic cells, stem cells and progenitor cells; and instructions for use. The at least one cell survival factor can be hVEGF. The at least one nucleic acid can further encode a second cell survival factor (e.g., hIGF-1). The at least one nucleic acid can be within a viral vector (e.g., within an rAAV vector). The subject may be one having ischemia or ischemia-related disease (e.g., PAD, CAD, ischemic heart disease, and heart failure). The tissue can be, for example, cardiac or skeletal tissue. The tissue can be infracted myocardium and the plurality of at least one of: somatic cells, stem cells, and progenitor cells can be delivered by intra-cardiac injection. The plurality of at least one of: somatic cells, stem cells, and progenitor cells can include MSCs. The hypoxia-regulated promoter can be a conditionally silenced promoter. The at least one nucleic acid encoding at least one cell survival factor can encode at least one of: VEGF, FGF, IGF-1, PDGF, and HIF-1. The plurality of at least one of: somatic cells, stem cells, and progenitor cells can be MSCs obtained from at least one of: bone marrow, adipose, skin, placenta, fetus, endothelial progenitor cells, CD34+ cells, hematopoietic cells, cardiac myoblasts, skeletal myoblasts, cardiac stem cells, skeletal stem cells, satellite cells, fibroblasts, myofibroblasts, smooth muscle cells, embryonic stem cells, and adult stem cells.

Unless otherwise defined, all technical terms used herein have the same meaning as commonly understood by one of ordinary skill in the art to which this invention belongs.

As used herein, a “nucleic acid” or a “nucleic acid molecule” means a chain of two or more nucleotides such as RNA (ribonucleic acid) and DNA (deoxyribonucleic acid), and chemically-modified nucleotides. A “purified” nucleic acid molecule is one that is substantially separated from other nucleic acid sequences in a cell or organism in which the nucleic acid naturally occurs (e.g., 30, 40, 50, 60, 70, 80, 90, 95, 96, 97, 98, 99, 100% free of contaminants). The terms include, e.g., a recombinant nucleic acid molecule incorporated into a vector, a plasmid, a virus, or a genome of a prokaryote or eukaryote. Examples of purified nucleic acids include cDNAs, micro-RNAs, fragments of genomic nucleic acids, nucleic acids produced polymerase chain reaction (PCR), nucleic acids formed by restriction enzyme treatment of genomic nucleic acids, recombinant nucleic acids, and chemically synthesized nucleic acid molecules. A “recombinant” nucleic acid molecule is one made by an artificial combination of two otherwise separated segments of sequence, e.g., by chemical synthesis or by the manipulation of isolated segments of nucleic acids by genetic engineering techniques.

By the term “gene” is meant a nucleic acid molecule that codes for a particular protein, or in certain cases, a functional or structural RNA molecule.

When referring to an amino acid residue in a peptide, oligopeptide or protein, the terms “amino acid residue”, “amino acid” and “residue” are used interchangably and, as used herein, mean an amino acid or amino acid mimetic joined covalently to at least one other amino acid or amino acid mimetic through an amide bond or amide bond mimetic.

As used herein, “protein” and “polypeptide” are used synonymously to mean any peptide-linked chain of amino acids, regardless of length or post-translational modification, e.g., glycosylation or phosphorylation.

By the phrase “growth and survival factors” is meant any gene product that confers cell growth and/or survival when expressed in a target tissue.

When referring to a nucleic acid molecule or polypeptide, the term “native” refers to a naturally-occurring (e.g., a wild-type (WT)) nucleic acid or polypeptide.

As used herein, the phrase “sequence identity” means the percentage of identical subunits at corresponding positions in two sequences (e.g., nucleic acid sequences, amino acid sequences) when the two sequences are aligned to maximize subunit matching, i.e., taking into account gaps and insertions. Sequence identity can be measured using sequence analysis software (e.g., Sequence Analysis Software Package from Accelrys CGC, San Diego, Calif.).

The phrases “isolated” or biologically pure” refer to material (e.g., nucleic acids, stem cells) which is substantially or essentially free from components which normally accompany it as found in its native state.

The term “labeled,” with regard to a nucleic acid, protein, probe or antibody, is intended to encompass direct labeling of the nucleic acid, protein, probe or antibody by coupling (i.e., physically or chemically linking) a detectable substance (detectable agent) to the nucleic acid, protein, probe or antibody.

By the term “progenitor cell” is meant any somatic cell which has the capacity to generate fully differentiated, functional progeny by differentiation and proliferation. In another embodiment, progenitor cells include progenitors from any tissue or organ system, including, but not limited to, blood, nerve, muscle, skin, gut, bone, kidney, liver, pancreas, thymus, and the like. Progenitor cells are distinguished from “differentiated cells,” which are defined in another embodiment, as those cells which may or may not have the capacity to proliferate, i.e., self-replicate, but which are unable to undergo further differentiation to a different cell type under normal physiological conditions. In one embodiment, progenitor cells are further distinguished from abnormal cells such as cancer cells, especially leukemia cells, which proliferate (self-replicate) but which generally do not further differentiate, despite appearing to be immature or undifferentiated.

As used herein, the term “totipotent” means an uncommitted progenitor cell such as embryonic stem cell, i.e., both necessary and sufficient for generating all types of mature cells. Progenitor cells which retain a capacity to generate all pancreatic cell lineages but which cannot self-renew are termed “pluripotent.” In another embodiment, cells which can produce some but not all endothelial lineages and cannot self-renew are termed “multipotent”.

As used herein, the phrase “bone marrow-derived progenitor cells” means progenitor cells that come from a bone marrow stem cell lineage. Examples of bone marrow-derived progenitor cells include bone marrow-derived (BM-derived) MSC and EPCs.

The term “homing” refers to the signals that attract and stimulate the cells involved in healing to migrate to sites of injury (e.g., to ischemic areas) and aid in repair (e.g, promote regeneration of vasculature, arteriogenesis).

By the phrases “therapeutically effective amount” and “effective dosage” is meant an amount sufficient to produce a therapeutically (e.g., clinically) desirable result; the exact nature of the result will vary depending on the nature of the disorder being treated. The compositions described herein can be administered from one or more times per day to one or more times per week. The skilled artisan will appreciate that certain factors can influence the dosage and timing required to effectively treat a subject, including but not limited to the severity of the disease or disorder, previous treatments, the general health and/or age of the subject, and other diseases present. Moreover, treatment of a subject with a therapeutically effective amount of the compositions and cells described herein can include a single treatment or a series of treatments.

As used herein, the term “treatment” is defined as the application or administration of a therapeutic agent (e.g., cells, a composition) described herein, or identified by a method described herein, to a patient, or application or administration of the therapeutic agent to an isolated tissue or cell line from a patient, who has a disease, a symptom of disease or a predisposition toward a disease, with the purpose to cure, heal, alleviate, relieve, alter, remedy, ameliorate, improve or affect the disease, the symptoms of disease, or the predisposition toward disease.

The terms “patient” “subject” and “individual” are used interchangeably herein, and mean a mammalian subject to be treated, with human patients being preferred. In some cases, the methods described herein find use in experimental animals, in veterinary applications, and in the development of animal models for disease, including, but not limited to, rodents including mice, rats, and hamsters, as well as non-human primates.

Although methods, compositions, cells, and kits similar or equivalent to those described herein can be used in the practice or testing of the present invention, suitable methods, compositions, cells, and kits are described below. All publications, patent applications, and patents mentioned herein are incorporated by reference in their entirety. In the case of conflict, the present specification, including definitions, will control. The particular embodiments discussed below are illustrative only and not intended to be limiting.

### BRIEF DESCRIPTION OF THE DRAWINGS

FIG. 1 is a series of micrographs of cells showing that gene therapy promotes stem cell survival. AAV9-CS-PGK-VEGF was delivered by i.m. injection. After 3 weeks limbs were made ischemic by ligation and excision of the femoral artery and ischemic muscle was injected with DiI-labeled syngenic mesenchymal stem cells (MSCs). Rabbits were sacrificed after 5 days and fluorescence visualized by confocal microscopy. Left 6 panels are MSCs alone+ischemia; right panels are MSCs+prior gene therapy+ischemia. MSC survival was >3-fold higher in the +gene therapy group (n=6; p<0.05).

FIG. 2 shows a series of photographs of blood vessels dermal tissue overlying ischemic muscle showing combined gene and stem cell therapy. Hind limbs were injected with AAV9 expressing VEGF under the direction of a hypoxia-regulated conditionally silenced promoter. After 3 weeks, ischemia was induced in the hind limb as in FIG. 1 and after another 48 h limbs were injected with syngeneic mesenchymal stem cells. (a) Top panel control subdermal tissue; 2nd top, ischemic tissue 1-week with PBS; 3rd top ischemia+AAV+MSC 1-week post-treatment; bottom ischemia+AAV+MSC 4 weeks post treatment (b). example of ulcerous skin overlying ischemic muscle.

FIG. 3 describes a second model of ischemia wherein tissue engineering with hypoxia-regulated conditionally silenced VEGF/IGF-1 combined with stem cell therapy can induce directional vessel growth and tissue salvage. Referring to FIGS. 3a-3d, diabetic db/db mice were subject to dermal+subdermal ischemia on the dorsal surface by creating longitudinal incisions and insertion of a silicon sheet under the skin to separate the skin from the underlying tissue (described in Chang et al, Circulation. 2007, 11; 116(24):2818-29). The skin is reapproximated with 6-0 nylon sutures, indicated by yellow arrowheads. Over a period of approximately 2 weeks there is progressive tissue necrosis that begins in the mid-regions of the sutured skin and in untreated animals extends over the entire region of the surgery and results in loss of the entire superficial dermus. FIG. 3d shows an example of a treated animal subjected to the same procedure but receiving treatment with gene therapy 3 days before ischemia using AAV-CS-hVEGF/IGF-1 (FROG/TOAD) with mesenchymal stem cell delivery at the time of ischemia. Animals that received the combined conditionally silenced gene therapy+stem cell therapy were protected and the tissue was salvaged. FIGS. 3e-3g show the order of blood vessels in this ischemia/regeneration/reperfusion model using wild type or db/db mice. Before surgery, vessels are typically oriented in a transverse direction across the dermus with respect to the spine (3e); several days after surgery when re-growth is possible new vessels grow in a longitudinal direction towards the central region of the dorsal surface where ischemia is the most severe, and the source of angiogenic and chemoattractant factors (3f). FIG. 3g shows an example of a light micrograph confirming the same effect; 3h shows central necrosis developing after 1-week in an untreated non-responsive mouse. Production of angiogenic and chemoattractant factors is compromised by diabetes but can be enhanced in an ischemia-dependent manner by hypoxia-regulated conditionally silenced gene/stem cell therapy. FIGS. 3i and 3j show the same effect measured by the Doppler technique. In FIG. 3i, immediately after surgery, blood flow is transverse with respect to the spine, whereas 3 days post surgery (3j) new vessels are transporting blood longitudinally in the direction of ischemia. FIG. 3k shows our proposed mechanism for combined gene and stem cell therapy for ischemia. The boxed area shows the region of intense ischemia of tissue that has been pre-engineered with hypoxia-regulated conditionally silenced VEGF/IGF-1. VEGF and IGF-1 genes are silent in normoxic tissue but are rapidly activated by ischemia to a level that is determined by the severity of ischemia. Activation of these angiogenic survival genes in the ischemic tissue protects the host tissue, activates angiogenesis and attracts host stem cells from the circulation providing a more conducive environment for cell and tissue survival. These tissue responses are suppressed when the host is diabetic. When new cells (stem cells, fibroblasts, skeletal myoblasts) are subsequently injected into the ischemic tissue as cell therapy, the survival of the injected cells is critically dependent on the environment within the ischemic tissue. In the methods described herein, tissue engineering with hypoxia-regulated conditionally silenced genes provides enhanced survival for injected cells as well as local and circulating host cells (vascular cells, fibroblasts, stem cells) that migrate towards the region of ischemic injury. A hypoxia-regulated conditionally silenced gene expression step is essential for safety and optimal responses of the gene, cells and growth/survival/chemoattractant factors.

FIG. 4 describes construction of the optimally regulated gene therapy vector for promoting cell survival, directional vessel growth and tissue salvage. The vector contains silencer elements NRSE (Neuronal Responsive Silencer Element)+HRE (Hypoxia Responsive Element) and FROG+TOAD+HRE. FROG and TOAD may be combined as FROG+TOAD+HRE or used separately as FROG+HRE or TOAD+HRE; HRE may be HIF-1 binding elements and may be substituted by metal response elements (MREs) (Murphy et al, Cancer Res. 1999 Mar. 15; 59(6):1315-22).

### DETAILED DESCRIPTION

The methods, compositions, cells and kits described herein are based on the discovery that stem cells, when injected into ischemic tissue of mammals, can be protected by preconditioning of the ischemic tissue with one or more hypoxia-regulated growth and survival factors (e.g., human VEGF (hVEGF) and human IGF-1 (hIGF-1)). Methods, compositions, cells and kits for treating tissue injured by ischemia or at risk of ischemic injury in a subject are thus described herein. The methods and compositions encompass (i) a procedure to safely engineer ischemic tissues by gene therapy and provide an environment that promotes survival of potentially therapeutic cells including stem cells contained within the ischemic tissue engineered in said manner, and (ii) a procedure wherein gene therapy with hypoxia-regulated conditionally silenced genes combined with cell therapy promotes directional growth of new blood vessels, reperfusion, and salvage of ischemic tissue

The below described preferred embodiments illustrate adaptations of these methods, compositions, cells, and kits. Nonetheless, from the description of these embodiments, other aspects of the invention can be made and/or practiced based on the description provided below.

### Biological Methods

Methods involving conventional molecular biology techniques are described herein. Such techniques are generally known in the art and are described in detail in methodology treatises such as Molecular Cloning: A Laboratory Manual, 3rd ed., vol. 1-3, ed. Sambrook et al., Cold Spring Harbor Laboratory Press, Cold Spring Harbor, N.Y., 2001; and Current Protocols in Molecular Biology, ed. Ausubel et al., Greene Publishing and Wiley-Interscience, New York, 1992 (with periodic updates). Conventional methods of gene transfer and gene therapy may also be adapted for use in the present invention. See, e.g., Gene Therapy Principles and Applications, ed. T. Blackenstein, Springer Verlag, 1999; and Gene Therapy Protocols (Methods in Molecular Medicine), ed. P.D. Robbins, Humana Press, 1997. Methods for culturing stem cells, progenitor cells and hematopoietic cells and for autologous progenitor/stem cell therapy are well known to those skilled in the art. See, e.g., Progenitor Cell Therapy for Neurological Injury (Stem Cell Biology and Regenerative Medicine), Charles S. Cox, ed., Humana Press, 1^{st} ed., 2010; A Manual for Primary Human Cell Culture (Manuals in Biomedical Research), Jan-Thorsten Schantz and Kee Woei Ng, World Scientific Publishing Co., 2^{nd} ed., 2010; and U.S. Pat. Nos. 7,790,458, 7,655,225, and 7,799,528.

### Compositions for Treating Ischemia

Compositions for treating ischemic diseases and ischemia-related diseases such as PAD and CAD are described herein. The compositions described herein can be used for treating any type of ischemia or ischemia-related disease or disorder, in addition to CAD and PAD, including wound healing, kidney, liver, intestinal, scalp, brain, lung ischemia, stroke, small vessel ischemic disease, subcortical ischemic disease, ischemic cerebrovascular disease, ischemic bowel disease, carotid artery disease, ischemic colitis, diabetic retinopathy, and various transplanted organs including pancreatic islets to treat diabetes. Such compositions generally include at least one nucleic acid encoding at least one cell survival factor for protecting stem and/or progenitor cells from ischemia in the subject. The at least one nucleic acid is operably-linked typically to a hypoxia-regulated, conditionally silenced promoter such that expression of the at least one cell survival factor is under the control of the hypoxia-regulated promoter. In some embodiments, the at least one nucleic acid is operably linked to a conditionally silenced promoter that is responsive to inflammation (e.g., a promoter containing at least one IRE), and in some cases, to a conditionally silenced promoter that is responsive to inflammation and hypoxia (ischemia), e.g., a promoter containing an IRE and at least one of: an HRE and a MRE. A conditionally silenced promoter as described herein can include or be operably linked to any suitable element that promotes or results in conditional silencing in ischemic tissue. Examples of such elements include HREs, IREs, and MREs. A conditionally silenced promoter as described herein can include or be operably linked to one or more of these elements (e.g., a combination of two or more of: HRE, MRE, and IRE). In addition to hypoxia-regulated promoters, inflammation-regulated promoters, and promoters responsive to both inflammation and hypoxia (ischemia), nucleic acids encoding at least one cell survival factor can be operably linked to constitutive promoters, tissue-specific promoters, shear and oxidative stress-regulated promoters, metal-regulated promoters, and inflammation-regulated promoters. Examples of cell survival factors include VEGF and IGF-1, FGF, hepatocyte growth factor (HGF), PDGF, SDF-1, heme oxygenase, HIF-1, erythropoietin, angiopoietin, Akt, proliferation-inducing ligand, cellular inhibitor of apoptosis protein (c-IAP1), c-IAP2, TNF receptor-associated factor-1 (TRAF-1), TRAF-2, B-cell leukemia/lymphoma-2 (Bcl-2), Bcl-x, A1, and cellular Fas-associated death domain (FADD)-like interleukin-1beta-converting enzyme-like inhibitory protein (c-FLIP), Pim-1, FoxO factors, Nmnat2, mTOR, Nerve Growth Factor (NGF), interleukins, anti-oxidants, and anti-inflammatory factors (IL-10). Any suitable cell survival factor(s), however, can be provided to the subject. In some embodiments, the at least one nucleic acid encodes two or more cell survival factors (e.g, both VEGF and IGF-1).

Other nucleic acid molecules as described herein include variants of the native genes encoding cell survival factors (e.g, VEGF and IGF-1) such as those that encode fragments, analogs and derivatives of a native cell survival factor protein. Such variants may be, e.g., a naturally occurring allelic variant of the native genes encoding cell survival factors (e.g, both VEGF and IGF-1), a homolog of the native genes encoding cell survival factors (e.g, both VEGF and IGF-1), or a non-naturally occurring variant of the native genes encoding cell survival factors (e.g, both VEGF and IGF-1). These variants have a nucleotide sequence that differs from the native genes in one or more bases. For example, the nucleotide sequence of such variants can feature a deletion, addition, or substitution of one or more nucleotides of the native genes encoding cell survival factors (e.g, VEGF and IGF-1).

In other embodiments, variant cell survival factor (e.g, VEGF and IGF-1) proteins displaying substantial changes in structure can be generated by making nucleotide substitutions that cause less than conservative changes in the encoded polypeptide. Examples of such nucleotide substitutions are those that cause changes in (a) the structure of the polypeptide backbone; (b) the charge or hydrophobicity of the polypeptide; or (c) the bulk of an amino acid side chain. Nucleotide substitutions generally expected to produce the greatest changes in protein properties are those that cause non-conservative changes in codons. Examples of codon changes that are likely to cause major changes in protein structure are those that cause substitution of (a) a hydrophilic residue, e.g., serine or threonine, for (or by) a hydrophobic residue, e.g., leucine, isoleucine, phenylalanine, valine or alanine; (b) a cysteine or proline for (or by) any other residue; (c) a residue having an electropositive side chain, e.g., lysine, arginine, or histadine, for (or by) an electronegative residue, e.g., glutamine or aspartine; or (d) a residue having a bulky side chain, e.g., phenylalanine, for (or by) one not having a side chain, e.g., glycine.

Naturally occurring allelic variants of native genes encoding cell survival factors (e.g, VEGF and IGF-1) or native mRNAs as described herein are nucleic acids isolated from human tissue that have at least 75% (e.g., 76%, 77%, 78%, 79%, 80%, 81%, 82%, 83%, 84%, 85%, 86%, 87%, 88%, 89%, 90%, 92%, 93%, 94%, 95%, 96%, 97%, 98%, and 99%) sequence identity with the native genes encoding cell survival factors (e.g, VEGF and IGF-1) or corresponding native mRNAs, and encode polypeptides having structural similarity to a native cell survival factor (e.g, VEGF and IGF-1) protein. Homologs of the native genes encoding cell survival factors (e.g, VEGF and IGF-1) or corresponding native mRNAs as described herein are nucleic acids isolated from other species that have at least 75% (e.g., 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, and 99%) sequence identity with the native human genes encoding cell survival factors (e.g, VEGF and IGF-1) or native corresponding human mRNAs, and encode polypeptides having structural similarity to native human cell survival factor (e.g, VEGF and IGF-1) proteins. Public and/or proprietary nucleic acid databases can be searched to identify other nucleic acid molecules having a high percent (e.g., 70, 80, 90% or more) sequence identity to the native genes encoding cell survival factors (e.g, VEGF and IGF-1) or corresponding native mRNAs. Non-naturally occurring genes encoding cell survival factors (e.g, VEGF and IGF-1) or mRNA variants are nucleic acids that do not occur in nature (e.g., are made by the hand of man), have at least 75% (e.g., 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, and 99%) sequence identity with the native human genes encoding cell survival factors (e.g, VEGF and IGF-1) or corresponding native human mRNAs, and encode polypeptides having structural similarity to native human cell survival factor (e.g, VEGF and IGF-1) proteins. These non-naturally occurring nucleic acids are encompassed by the methods, compositions, cells and kits described herein.

### Therapeutic Stem and/or Progenitor Cells

Adult stem/progenitor cells may be obtained directly from the bone marrow (for example, from posterior iliac crests), any other tissue, or from peripheral blood. Isolated stem cells and progenitor cells can be maintained and propagated in any appropriate cell culture growth medium. Standardized procedures for the isolation, enrichment and storage of stem/progenitor cells are well known in the art. Methods for culturing stem cells, progenitor cells, and hematopoietic cells are known to those skilled in the art.

The cells which are employed may be fresh, frozen, or have been subjected to prior culture. They may be fetal, neonate, adult. Hematopoietic cells may be obtained from fetal liver, bone marrow, blood, cord blood or any other conventional source. The progenitor and/or stem cells can be separated from other cells of the hematopoietic or other lineage by any suitable method.

Marrow samples may be taken from patients with ischemic disease (e.g., CAD, PAD), and enriched populations of hematopoietic stem and/or progenitor cells isolated by any suitable means (e.g., density centrifugation, counterflow centrifugal elutriation, monoclonal antibody labeling and fluorescence activated cell sorting). The stem and/or progenitor cells in this cell population can then be administered to a subject in need following administration to the subject of a composition including at least one nucleic acid encoding at least one cell survival factor for protecting stem and/or progenitor cells from ischemia in the subject, wherein the at least one nucleic acid is operably linked to a hypoxia-regulated and/or conditionally silenced promoter such that expression of the at least one cell survival factor is under the control of the hypoxia-regulated promoter.

Methods for extracting and culturing somatic cells from multiple tissues including skeletal muscle, liver, neuronal, blood vessels, and other organs are known to those skilled in the art.

### Methods of Stem Cell Therapy

Methods of stem cell therapy involving administration of stem cells as well as a composition that protects the stem cells from ischemia are described herein. Examples of such therapeutic methods include methods of treating tissue injured by ischemia or at risk of ischemic injury. A typical method of treating tissue injured by ischemia or at risk of ischemic injury in a subject includes: administering to the subject a therapeutically effective amount of a composition including at least one nucleic acid encoding at least one cell survival factor for protecting stem and/or progenitor cells from ischemia in the subject, the at least one nucleic acid operably linked to a hypoxia-regulated promoter; and subsequently administering to the subject a therapeutically effective amount of stem and/or progenitor cells. Administering the at least one nucleic acid followed by administration of the stem and/or progenitor cells induces directional growth of blood vessels and arteriogenesis at one or more sites of ischemia or ischemic injury in the subject. The stem and/or progenitor cells can be administered at any suitable time point concomitant with or subsequent to administration of the at least one nucleic acid. For example, the stem and/or progenitor cells can be administered simultaneously with the nucleic acid or between 0 and 24 h or at any time up to 12 months subsequent to administration of the at least one nucleic acid. For example, cells (including stem cells) would ideally be administered after gene expression by said nucleic acid is activated and accumulation of gene product (typically 4 hours to 7 days after ischemia and 4 h to 12 months after delivery of nucleic acid). The time period for administration of cells is variable because ischemia may re-occur months or even years after administration of nucleic acid. When ischemia occurs in tissue containing the at least one nucleic acid at any time after its administration, the gene product (e.g., VEGF, IGF-1) will accumulate and be available for cell protection angiogenesis, arteriogenesis and tissue salvage.

The methods described herein can be used to treat any disease or condition associated with ischemia or ischemic injury. Examples of conditions or diseases associated with ischemic injury include PAD and CAD. Thus, one embodiment of a method of treating tissue injured by ischemia or at risk of ischemic injury in a subject involves treating PAD or CAD in a subject. In some methods, a plurality of bone marrow-derived progenitor cells and/or stem cells and somatic (e.g., non-stem somatic) cells (e.g., MSCs from multiple sources including but not limited to: bone marrow, adipose, skin, fetal, placental, embryonic stem cell derived, EPCs (e.g., CD34+/CD133+/CD31+ EPCs), mixed bone marrow or blood derived lineage negative (Lin−) cells, bone marrow or blood derived mixed mononuclear cells, fibroblasts, smooth muscle cells, skeletal myoblasts and satellite myocytes, cardiac stem cells, etc.) are administered to the subject in an amount effective to promote directional growth of blood vessels and arteriogenesis in one or more areas of ischemia in the subject. In such an embodiment, the progenitor cells and/or stem cells are administered to the subject following administration to the subject of a composition including at least one nucleic acid encoding at least one cell survival factor for protecting stem and/or progenitor cells from ischemia in the subject, such that expression of the at least one cell survival factor is under control of a hypoxia-regulated promoter, and the progenitor cells and/or stem cells are protected from ischemia.

In these methods, the at least one nucleic acid can be administered to a subject by any suitable method or route. In a typical embodiment, the nucleic acid is delivered to the subject via a vector (e.g. a nucleic acid expression vector). Many vectors useful for transferring exogenous genes into target mammalian cells are available. The at least one nucleic acid can be included within a viral vector, for example. Typically, a viral vector is encompassed within a virion (or particle) and the vector-containing virion or particle is administered to or contacted with a cell. In the experiments described below, rAAV vectors were used to deliver the at least one nucleic acid encoding a cell survival factor (e.g., hVEGF, IGF-1) to mammalian subjects. However, any suitable vector may be used. When using rAAV, for example, any suitable AAV serotype may be used; AAV serotypes 1-9 have been shown to express well in skeletal and cardiac muscles although with varying efficiency. Examples of suitable serotypes include the following: AAV1, 2, 5-8, shown to express efficiently in heart (Palomequel et al, Gene Therapy (2007) 14, 989-997), and serotypes 2, 7-9 shown to transduce skeletal muscles (Evans et al, Metabolism. 2011, 60(4):491-8). For neuronal targets, AAV1, 2, 6, 7 and 9 were shown to efficiently infect hypocampal and cortical neurons (Royo et al, Molecular Therapy (2006) 13, S347), and rAAV hybrid serotypes rAAV 2/1, 2/5, 2/8 and rAAV2/2 were also shown to be effective in neuronal transduction again with some differences in efficiency (McFarland et al, J Neurochem. 2009 109(3): 838-845). For liver transduction, serotypes AAV8, AAVhu.37, and AAVrh.8 were shown to be the most efficient (Wang et al, Molecular Therapy, 18, 118-125, 2010). AAV serotype 4 was shown to be tropic for kidney, lung and heart (Zincarelli et al, Molecular Therapy (2008) 16 6, 1073-1080). AAV1 and AAV8 were shown to be more efficient than AAV2 and AAV6, respectively, for transduction of pancreatic islets and beta-cells (Loilet et al, Gene Therapy (2003) 10, 1551-1558; Wang et al, Diabetes, 2006 vol. 55 no. 4, 875-884). In addition to the natural tissue tropism of specific rAAV serotypes, further tissue-specificity can be achieved by using tissue-specific promoters and/or incorporating coding sequences for expressing peptides that recognize cell-specific epitopes. The vectors may be episomal, e.g. plasmids, virus derived vectors such cytomegalovirus, adenovirus, etc., or may be integrated into the target cell genome, through homologous recombination or random integration, e.g. retrovirus derived vectors such MMLV, HIV-1, ALV, lentivirus etc. Various techniques using viral vectors for the introduction of nucleic acids into mammalian cells are provided for according to the methods, compositions, cells and kits described herein. Viruses are naturally evolved vehicles which efficiently deliver their genes into host cells and therefore are desirable vector systems for the delivery of therapeutic nucleic acids. Preferred viral vectors exhibit low toxicity to the host cell and produce/deliver therapeutic quantities of the nucleic acid of interest (in a typical embodiment, in a regulated, conditional manner). Retrovirus based vectors (e.g., see Baum et al. (1996) J Hematother 5(4):323-9; Schwarzenberger et al. (1996) Blood 87:472-478; Nolta et al. (1996) P.N.A.S. 93:2414-2419; and Maze et al. (1996) P.N.A.S. 93:206-210) and lentivirus vectors may find use within the methods described herein (e.g., see Mochizuki et al. (1998) J Virol 72(11):8873-83). The use of adenovirus-based vectors has also been characterized, (e.g. see Ogniben and Haas (1998) Recent Results Cancer Res 144:86-92). Viral vector methods and protocols are reviewed in Kay et al. Nature Medicine 7:33-40, 2001.

Also in these methods, the therapeutic stem and/or progenitor cells can be administered to a subject by any suitable route, e.g., intravenously, or directly to a target site. Several approaches may be used for the introduction of stem and/or progenitor cells into the subject, including catheter-mediated delivery I.V. (e.g., endovascular catheter), or direct injection into a target site. Techniques for the isolation of autologous stem cells or progenitor cells and transplantation of such isolated cells are known in the art. Microencapsulation of cells, for example, is another technique that may be used. Autologous as well as allogeneic cell transplantation may be used according to the invention.

The therapeutic methods described herein in general include a combination therapy which involves administration of a therapeutically effective amount of the compositions and cells described herein to a subject (e.g., animal, human) in need thereof, including a mammal, particularly a human. Such treatment will be suitably administered to subjects, particularly humans, suffering from, having, susceptible to, or at risk for a disease, disorder, or symptom thereof. Determination of those subjects “at risk” can be made by any objective or subjective determination by a diagnostic test or opinion of a subject or health care provider. The methods and compositions herein may be used in the treatment of any other disorders in which ischemia or ischemia-related conditions may be implicated.

In one embodiment, a method of treating an ischemia-related disease or disorder (e.g., PAD or CAD) in a subject includes monitoring treatment progress. Monitoring treatment progress in a subject generally includes determining a measurement of, for example, vasculogenesis, vasculature, arteriogenesis, or tissue damage at the site of injury (ischemic injury) or other diagnostic measurement in a subject having an ischemia-related disease, prior to administration of a therapeutic amount of a composition sufficient for protecting stem and/or progenitor cells in an ischemic environment followed by administration of a therapeutic amount of stem and/or progenitor cells sufficient to increase directional growth of blood vessels and arteriogenesis at the site of injury in the subject. At one or more time points subsequent to the subject having been administered a therapeutic amount of a composition sufficient for protecting stem and/or progenitor cells in an ischemic environment and a therapeutic amount of stem and/or progenitor cells sufficient to increase directional growth of blood vessels and arteriogenesis at the site of injury, a second measurement of vasculogenesis, vasculature, arteriogenesis, or tissue damage at the site of injury is determined and compared to the first measurement of vasculogenesis, vasculature, arteriogenesis, or tissue damage. The first and subsequent measurements are compared to monitor the course of the disease and the efficacy of the therapy.

### Kits

Described herein are kits for treating ischemia and/or an ischemia-related disease or disorder (e.g., PAD or CAD) in a mammalian subject. A typical kit includes a therapeutically effective amount of a composition including at least one nucleic acid encoding at least one cell survival factor for protecting stem and/or progenitor cells from ischemia in the subject, the at least one nucleic acid operably linked to a hypoxia-regulated promoter, and a therapeutically effective amount of stem and/or progenitor cells with instructions for administering the composition and the cells to the subject. The cells can be packaged by any suitable means for transporting and storing cells; such methods are well known in the art. The instructions generally include one or more of: a description of the composition and the cells; dosage schedule and administration for treatment of ischemia and ischemia-related disorders (e.g., PAD, CAD); precautions; warnings; indications; counter-indications; overdosage information; adverse reactions; animal pharmacology; clinical studies; and/or references. The instructions may be printed directly on the container (when present), or as a label applied to the container, or as a separate sheet, pamphlet, card, or folder supplied in or with the container. Generally, a kit as described herein also includes packaging. In some embodiments, the kit includes a sterile container which contains a therapeutic or prophylactic composition; such containers can be boxes, ampules, bottles, vials, tubes, bags, pouches, blister-packs, or other suitable container forms known in the art. Such containers can be made of plastic, glass, laminated paper, metal foil, or other materials suitable for holding cells or medicaments.

### Administration of Compositions

The compositions and cells described herein may be administered to mammals (e.g., rodents, humans) in any suitable formulation. A description of exemplary pharmaceutically acceptable carriers and diluents, as well as pharmaceutical formulations, can be found in Remington's Pharmaceutical Sciences, a standard text in this field, and in USP/NF. Other substances may be added to the compositions to stabilize and/or preserve the compositions.

The compositions and cells of the invention may be administered to mammals by any conventional technique. The compositions and cells may be administered directly to a target site by, for example, surgical delivery to an internal or external target site, or by catheter (e.g., endovascular catheter) to a site accessible by a blood vessel. When treating a subject having, for example, PAD or CAD, the composition and cells may be administered to the subject intravenously, directly into cardiovascular tissue or arterial tissue, or to the surface of cardiovascular or arterial tissue. The compositions may be administered in a single bolus, multiple injections, or by continuous infusion (e.g., intravenously, by peritoneal dialysis, pump infusion). For parenteral administration, the compositions are preferably formulated in a sterilized pyrogen-free form. In a typical embodiment, a composition including at least one nucleic acid encoding at least one cell survival factor for protecting stem and/or progenitor cells from ischemia in the subject, the at least one nucleic acid operably linked to a hypoxia-regulated promoter for protecting stem and/or progenitor cells from ischemia is administered to the subject prior to administration of therapeutic stem and/or progenitor cells.

### Effective Doses

The compositions and cells described herein are preferably administered to a mammal (e.g., human) in an effective amount, that is, an amount capable of producing a desirable result in a treated mammal (e.g., preventing or treating ischemic conditions such as CAD or PAD, inducing directional growth of blood vessels and arteriogenesis). Such a therapeutically effective amount can be determined according to standard methods. Toxicity and therapeutic efficacy of the compositions utilized in methods of the invention can be determined by standard pharmaceutical procedures. As is well known in the medical and veterinary arts, dosage for any one subject depends on many factors, including the subject's size, body surface area, age, the particular composition to be administered, time and route of administration, general health, and other drugs being administered concurrently.

### EXAMPLES

The present invention is further illustrated by the following specific examples. The examples are provided for illustration only and should not be construed as limiting the scope of the invention in any way.

### Example 1

### Increased Stem Cell Survival by Gene Therapy

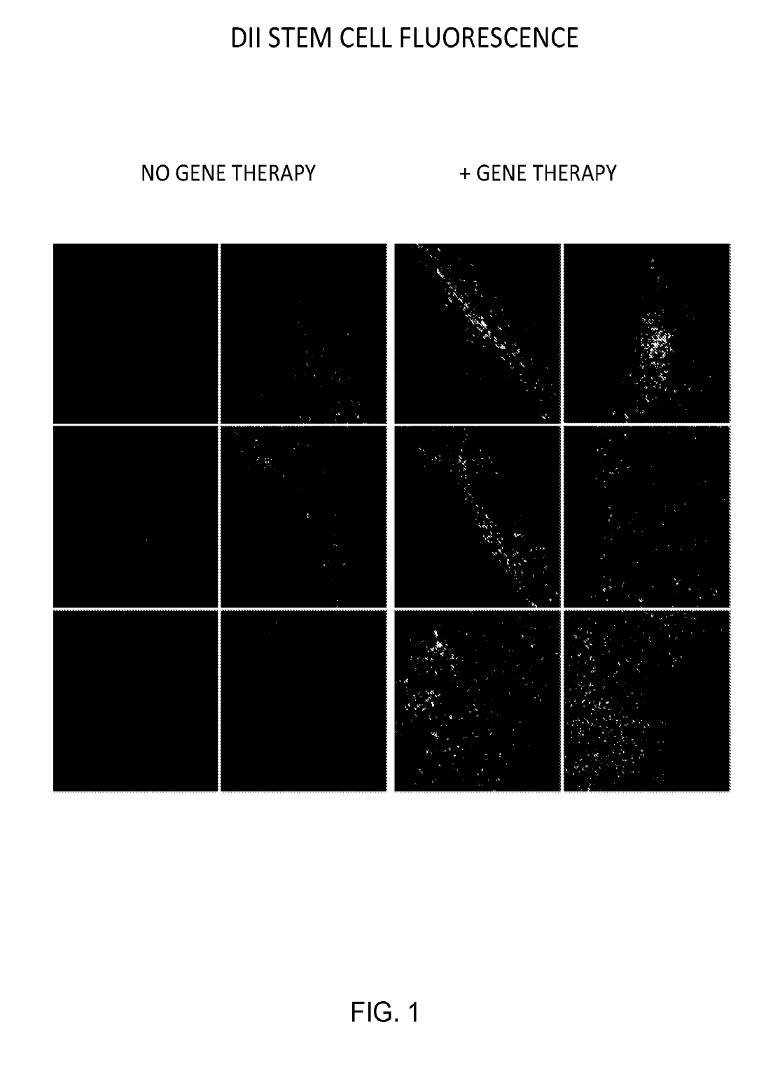

A rabbit hind limb ischemia model was used to determine whether VEGF gene delivery to ischemic hind limbs prior to stem cell delivery protected co-localized stem cells. Rabbit hind limbs (3 per group) were injected with 10⁻¹⁰ pfu AAV9-CS-VEGF (hypoxia-regulated conditionally silenced (CS) (or PBS) at 8 sites. After 3 weeks, ischemia was induced by femoral artery ligation and excision, and 2×10⁻⁵ DiI-labeled syngeneic rabbit MSCs were injected at the same sites as the genes, 48 h after surgery, a time that coincides with VEGF gene activation by ischemia. Rabbits were sacrificed after 5 more days, muscles sectioned through the injection sites and examined by confocal fluorescence microscopy for DiI-positive cells. FIG. 1 shows examples of fields with the maximum cell numbers from each group. Examination of 6 fields from 3 rabbits per group revealed >3-fold greater fluorescent cells in the gene therapy group (p<0.05). This is the first demonstration that regulated gene therapy can be used to enhance survival of stem cells in diseased (ischemic) muscle.

### Example 2

### Gene and Stem Cell Therapy Protect Against Ischemic Ulcers by Enhancing New Vessel Production

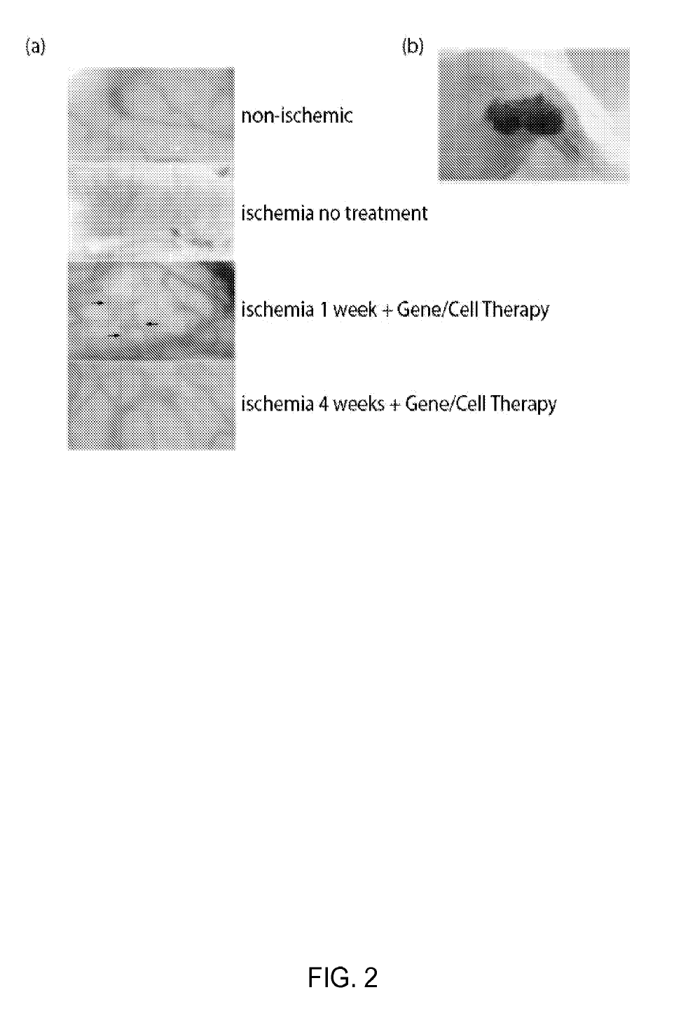

Many rabbits with hind limb ischemia develop ulcers in the skin overlying the ischemic muscle even when gene therapy is implemented. To determine whether ulcers were prevented by combined gene and stem cell therapy rabbits were treated as described in FIG. 1 ±gene/MSC treatments and examined at 1 and 4 weeks after gene/cell delivery. It was found that the combined gene and stem cell treatments eliminated ulcer formation and promoted increased vascularity of the sub-dermal tissues overlying the ischemic muscle (FIG. 2a). An example of an ulcer is shown in FIG. 2(b).

### Example 3

### Gene and Stem Cell Therapy for Wound Healing to Protect Dermal Tissue from Ischemia-Induced Necrosis

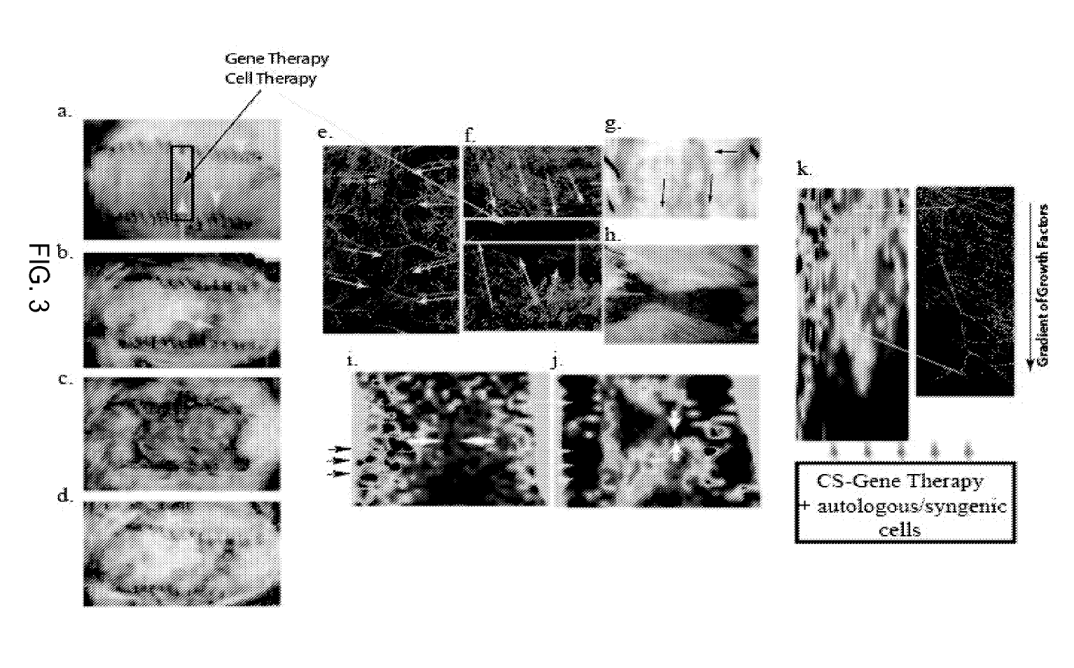

Diabetic db/db mice were subject to dermal/subdermal ischemia on the dorsal surface by making longitudinal skin incisions and inserting a silicon sheet under the skin (see Chang et al, Circulation. 2007, 11; 116(24):2818-29). The skin was reapproximated with 6-0 nylon sutures (indicated by yellow arrowheads). Necrosis begins in the mid-regions of the sutured skin and in untreated animals extends over the entire region of the surgery and results in loss of the entire superficial dermus (FIGS. 3a-3c). In FIG. 3d the dermus was injected with AAV-CS-hVEGF/IGF-1 (FROG/TOAD) (6× injection sites 5×10⁻⁹ genomes total) 3 days before ischemia Immediately after ischemia the same region received 10-4 syngenic bone marrow mesenchymal stem cells. Animals treated as in (3d) were protected and the tissue was salvaged (n=3). FIGS. 3e-3g show the order of blood vessels in this ischemia/regeneration/reperfusion model using wild type or db/db mice. Before surgery vessels were oriented in a transverse direction across the dermus with respect to the spine (3e); several days after surgery new vessels grow in a longitudinal direction towards the central region of the dorsal surface where ischemia is the most severe (3f). FIG. 3g shows an example of a light micrograph confirming the same effect; FIG. 3h shows central necrosis developing after 1-week in an untreated non-responsive mouse. FIGS. 3i and 3j show the same effect measured by the Doppler technique. In FIG. 3i, immediately after surgery, blood flow is transverse with respect to the spine, whereas 3 days post surgery (3j) new vessels are transporting blood longitudinally in the direction of ischemia. FIG. 3k shows a proposed mechanism for combined gene and stem cell therapy for ischemia. In the boxed area intense ischemia activates expression of AAV-CS-hVEGF/IGF-1 delivered 3-days prior to ischemia in a silenced form. Gene activation (1) protects endogenous host tissues (2) activates angiogenesis (2) enhances the production and secretion of survival factors and chemoattractant factors (3) enhances homing of host stem cells from the circulation (4) provides a more conducive environment survival of exogenous and endogenous stem and somatic cells. When new cells (e.g. stem cells, fibroblasts, skeletal myoblasts) are subsequently injected into the ischemic tissue these cells are also protected and synergize with endogenous cells to amplify all responses. In the methods described herein, tissue engineering with AAV-CS-hVEGF/IGF-1 provides enhanced survival for injected cells as well as local and circulating host cells (vascular cells, fibroblasts, stem cells) that migrate towards the region of ischemic injury. Conditionally silenced gene expression step is essential for safety and optimal responses of the gene, cells and growth/survival/chemoattractant factors.

In conclusion it has been shown that gene therapy with hypoxia-regulated AAV-VEGF provides enhanced stem cell survival when genes and cells are co-localized in ischemic tissue, increased vascularization of the skin overlying the ischemic muscles, protection against skin ulcers, and enhanced survival of dermal and subdermal tissues subjected to ischemia. This is the first evidence that combined gene and stem cell therapy works synergistically to enhance stem cell survival and promote revascularization and survival of ischemic tissue.

### Example 4

### Sequences of the FROG and TOAD Elements

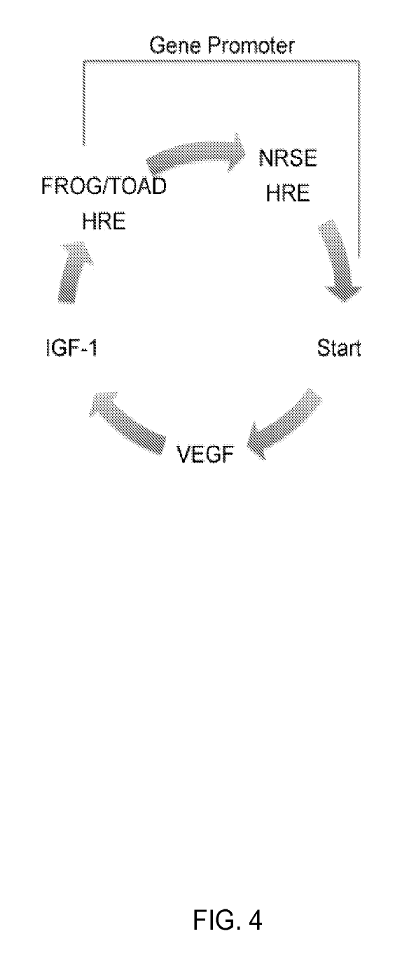

These elements are arranged in tandem at any location up to 5 kB upstream of the transcription start site of a gene promoter. The elements may also be arranged at multiple locations with respect to each other within the 5 kB sequence. Referring to FIG. 4, the hypoxia-regulated conditionally silenced promoter directs expression of VEGF and or IGF-1 genes positioned downstream of the transcription start site. In addition to the properties described in FIGS. 1-3, this vector was found to promote significantly improved tissue salvage in the mouse hind limb ischemia model compared with a vector containing only NRSE silencer and HRE elements. In practice any gene or number of genes expressing other survival/growth/pro-angiogenic or arteriogenic functions that promote blood vessel growth and/or tissue and cell survival can replace these genes. The most effective gene therapy for ischemic tissue engineering includes combinations of NRSE and FROG/TOAD elements with HREs or MREs. It was found that NRSE+FROG/TOAD conferred conditional silencing to multiple cell types including stem cells and neuronal cell that was not achieved by NRSE/HRE alone.

TOAD/PGK (Sense):
(SEQ ID NO: 1)
5′-CCGGCTCTTCCAGAGCAAGGCAACCACAGGAGACCCTGTCACGTCC
TGCACGACCTCTTCCAGAGCAAGGCAACCACAGGAGACCCTGTCACGTC
CTGCACGACCTCTTCCAGAGCAAGGCAACCACAGGAGACCCTGTCACGT
CCTGCACGAC-3′
TOAD/PGK (Antisense):
(SEQ ID NO: 2)
3′-GAGAAGGTCTCGTTCCGTTGGTGTCCTCTGGGACAGTGCAGGACGT
GCTGGAGAAGGTCTCGTTCCGTTGGTGTCCTCTGGGACAGTGCAGGACG
TGCTGGAGAAGGTCTCGTTCCGTTGGTGTCCTCTGGGACAGTGCAGGAC
GTGCTGGGCC-5′
FROG/PGK (Sense):
(SEQ ID NO: 3)
5′-CCGGGGTGTGCATTTAGCTAAATTCCCCACTGTCACGTCCTGCACG
ACGGTGTGCATTTAGCTAAATTCCCCACTGTCACGTCCTGCACGAC
GGTGTGCATTTAGCTAAATTCCCCACTGTCACGTCCTGCACGAC-3′
FROG/PGK (Antisense):
(SEQ ID NO: 4)
3′-CCACACGTAAATCGATTTAAGGGGTGACAGTGCAGGACGTGCTGCC
ACACGTAAATCGATTTAAGGGGTGACAGTGCAGGACGTGCTGCCACACG
TAAATCGATTTAAGGGGTGACAGTGCAGGACGTGCTGGGCC-5′
FROG-TOAD/PGK (Sense):
(SEQ ID NO: 5)
5′-CCGGCTCTTCCAGAGCAAGGCAACCACAGGAGACCCTGTCACGTCC
TGCACGACGGTGTGCATTTAGCTAAATTCCCCACTGTCACGTCCTGCAC
GACCTCTTCCAGAGCAAGGCAACCACAGGAGACCCTGTCACGTCCTGCA
CGACGGTGTGCATTTAGCTAAATTCCCCACTGTCACGTCCTGCACGA
C-3′
FROG-TOAD/PGK (Antisense):
(SEQ ID NO: 6)
3′-GAGAAGGTCTCGTTCCGTTGGTGTCCTCTGGGACAGTGCAGGACGT
GCTGCCACACGTAAATCGATTTAAGGGGTGACAGTGCAGGACGTGCTGG
AGAAGGTCTCGTTCCGTTGGTGTCCTCTGGGACAGTGCAGGACGTGCTG
CCACACGTAAATCGATTTAAGGGGTGACAGTGCAGGACGTGCTGGGC
C-5′

The sequences above are sequences of oligonucleotides encoding 3× repeat sequences of TOAD+HRE, FROG+HRE and combined FROG+TOAD+HRE. Single or multiple copies of these oligonucleotides are inserted alone or in combination with NRSE-HRE into AAV shuttle vectors upstream of a gene promoter such as the glycolytic enzyme phosphoglycerate kinase to confer conditional silencing of an expressed nucleic acid sequence such as VEGF and IGF-1. The combined use of FROG+TOAD+NRSE is required to obtain efficient conditional silencing in all cell types including muscle cells, fibroblasts, neuronal cells and stem cells.

### OTHER EMBODIMENTS

Any improvement may be made in part or all of the compositions, cells, kits, and method steps. All references, including publications, patent applications, and patents, cited herein are hereby incorporated by reference. The use of any and all examples, or exemplary language (e.g., “such as”) provided herein, is intended to illuminate the invention and does not pose a limitation on the scope of the invention unless otherwise claimed. Any statement herein as to the nature or benefits of the invention or of the preferred embodiments is not intended to be limiting, and the appended claims should not be deemed to be limited by such statements. More generally, no language in the specification should be construed as indicating any non-claimed element as being essential to the practice of the invention. In addition to nucleic acid (e.g., vector)-containing compositions, compositions as described herein can contain stem cells. This invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contraindicated by context.

What is claimed is:

1. A method of treating tissue injured by ischemia or at risk of ischemic injury in a subject, the method comprising the steps of:
a) administering to the subject a therapeutically effective amount of a composition comprising at least one nucleic acid encoding at least one cell survival factor for protecting one or more cell types selected from the group consisting of: somatic cells, stem cells, and progenitor cells, from ischemia in the subject, the at least one nucleic acid operably linked to a hypoxia-regulated promoter; and
b) administering to the subject a therapeutically effective amount of a plurality of at least one of: somatic cells, stem cells, and progenitor cells,
wherein administering the at least one nucleic acid followed by administration of the plurality of at least one of: somatic cells, stem cells, and progenitor cells induces directional growth of blood vessels and arteriogenesis at one or more sites of ischemia, ischemic injury, and potential ischemic injury in the subject.

2. The method of claim 1, wherein the at least one cell survival factor is human VEGF (hVEGF).

3. The method of claim 2, wherein the at least one nucleic acid further encodes a second cell survival factor.

4. The method of claim 3, wherein the second cell survival factor is human IGF-1 (hIGF-1).

5. The method of claim 1, wherein the at least one nucleic acid is comprised within a recombinant Adeno-Associated Virus (rAAV) vector.

6. The method of claim 1, wherein the subject has ischemia or ischemia-related disease.

7. The method of claim 6, wherein the ischemia-related disease is one selected from the group consisting of: peripheral artery disease (PAD), coronary artery disease (CAD), ischemic heart disease, and heart failure.

8. The method of claim 1, wherein the tissue is cardiac or skeletal tissue.

9. The method of claim 8, wherein the tissue is infracted myocardium and the plurality of at least one of: somatic cells, stem cells, and progenitor cells is delivered by intra-cardiac injection.

10. The method of claim 1, wherein the plurality of at least one of: somatic cells, stem cells, and progenitor cells comprises mesenchymal stem cells.

11. The method of claim 1, wherein the hypoxia-regulated promoter is a conditionally silenced promoter.

12. The method of claim 1, wherein the hypoxia-regulated promoter is conditionally silenced by a Neuronal Response Silencer Element (NRSE) and a Hypoxia Responsive Element (HRE).

13. The method of claim 1, wherein the hypoxia-regulated promoter is conditionally silenced by FROG and an HRE.

14. The method of claim 1, wherein the hypoxia-regulated promoter is conditionally silenced by TOAD and an HRE.

15. The method of claim 1, wherein the hypoxia-regulated promoter is conditionally silenced by FROG, TOAD, and an HRE.

16. The method of any of claims 12-15, wherein the hypoxia-regulated promoter is conditionally silenced by one or more combinations of: NRSE and HRE; FROG and HRE; TOAD and HRE; and FROG, TOAD and HRE.

17. The method of claim 11, wherein the hypoxia-regulated conditionally silenced promoter comprises at least one of: a metal response element (MRE) and an HRE, and optionally an inflammatory responsive element (IRE).

18. The method of claim 17, wherein the hypoxia-regulated conditionally silenced promoter comprises an HRE, an MRE, and an IRE, and is responsive to both hypoxia and inflammation.

19. The method of claim 1, wherein the at least one nucleic acid encoding at least one cell survival factor encodes at least one selected from the group consisting of: VEGF, FGF, IGF-1, PDGF, and HIF-1.

20. The method of claim 1, wherein the at least one of stem cells and progenitor cells are mesenchymal stem cells obtained from at least one selected from the group consisting of: bone marrow, adipose, endothelial progenitor cells, CD34+ cells, hematopoietic cells, cardiac myoblasts, skeletal myoblasts, cardiac stem cells, skeletal stem cells, satellite cells, fibroblasts, myofibroblasts, smooth muscle cells, embryonic stem cells, and adult stem cells.

21. The method of claim 1, wherein the tissue injured by ischemia or at risk of ischemic injury is selected from the group consisting of: skeletal muscle, cardiac muscle, kidney, liver, dermal tissue, scalp, and eye.

22. A method of treating tissue injured by ischemia or at risk of ischemic injury in a subject, the method comprising the steps of:
a) administering to the subject a therapeutically effective amount of a composition comprising at least one nucleic acid encoding at least one cell survival factor for protecting one or more cell types selected from the group consisting of: somatic cells, stem cells, and progenitor cells, from ischemia in the subject, the at least one nucleic acid operably linked to an inflammation-responsive promoter,
wherein the inflammation-responsive promoter comprises at least one IRE and optionally, an HRE; and
b) administering to the subject a therapeutically effective amount of a plurality of at least one of: somatic cells, stem cells, and progenitor cells,
wherein administering the at least one nucleic acid followed by administration of the plurality of at least one of: somatic cells, stem cells, and progenitor cells induces directional growth of blood vessels and arteriogenesis at one or more sites of ischemia, ischemic injury, and potential ischemic injury in the subject.

23. A kit for treating tissue injured by ischemia or at risk of ischemic injury in a mammalian subject, the kit comprising:
(a) a therapeutically effective amount of a composition comprising at least one nucleic acid encoding at least one cell survival factor for protecting at least one of somatic cells, stem cells and progenitor cells from ischemia in the subject, the at least one nucleic acid operably linked to a hypoxia-regulated promoter;
(b) a therapeutically effective amount of the at least one of somatic cells, stem cells and progenitor cells; and
(c) instructions for use.

24. The kit of claim 23, wherein the at least one cell survival factor is hVEGF.

25. The kit of claim 24, wherein the at least one nucleic acid further encodes a second cell survival factor.

26. The kit of claim 25, wherein the second cell survival factor is hIGF-1.

27. The kit of claim 23, wherein the at least one nucleic acid is comprised within an rAAV vector.

28. The kit of claim 23, wherein the subject has ischemia or ischemia-related disease.

29. The kit of claim 28, wherein the ischemia-related disease is one selected from the group consisting of: PAD, CAD, ischemic heart disease, and heart failure.

30. The kit of claim 23, wherein the tissue is cardiac or skeletal tissue.

31. The kit of claim 30, wherein the tissue is infracted myocardium and the plurality of at least one of: somatic cells, stem cells, and progenitor cells is delivered by intra-cardiac injection.

32. The kit of claim 23, wherein the plurality of at least one of: somatic cells, stem cells, and progenitor cells comprises mesenchymal stem cells.

33. The kit of claim 23, wherein the hypoxia-regulated promoter is a conditionally silenced promoter.

34. The kit of claim 23, wherein the at least one nucleic acid encoding at least one cell survival factor encodes at least one selected from the group consisting of: VEGF, FGF, IGF-1, PDGF, and HIF-1.

35. The kit of claim 23, wherein the plurality of at least one of: somatic cells, stem cells, and progenitor cells are mesenchymal stem cells obtained from at least one selected from the group consisting of: bone marrow, adipose, skin, placenta, fetus, endothelial progenitor cells, CD34+ cells, hematopoietic cells, cardiac myoblasts, skeletal myoblasts, cardiac stem cells, skeletal stem cells, satellite cells, fibroblasts, myofibroblasts, smooth muscle cells, embryonic stem cells, and adult stem cells.
